(19) United States
(12) Patent Application Publication
(10) Pub. No.: US 2005/0143685 A1
(43) Pub. Date: Jun. 30, 2005

(54) Drug dispensing components

(76) Inventors: Isabelle Denise Peyron, Evreux (FR); Hirotsugu Yasuda, Columbia, MO (US)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/490,240

(22) PCT Filed: Sep. 19, 2002
(86) PCT No.: PCT/GB02/04276

(30) Foreign Application Priority Data
Sep. 21, 2001 (GB) 0122725.5

(51) Int. Cl. A61N 1/30

(52) U.S. Cl. 604/19

(57) ABSTRACT

A medicament dispenser comprising a canister and a drug dispensing valve, wherein one or more surfaces of said canister and/or valve has a fluorinated coating provided by a process comprising generating one or more fluorine-containing radical species and polymerising said radicals on said one or more surfaces, provided that the radicals are not generated by a process involving continuous wave radio frequency plasma polymerisation is disclosed.

### RELATED APPLICATIONS

The present application claims priority from UK patent application No. 0122725.5 filed Sep. 21, 2001, the entire content of which is hereby incorporated herein by reference.

### FIELD OF THE INVENTION

The present invention relates to medicament dispensers, metered dose inhalers and components thereof. More especially, the invention relates to medicament dispensers and metered dose inhalers for consistently dispensing a prescribed dose of medicament.

### BACKGROUND TO THE INVENTION

Drugs for treating respiratory and nasal disorders are frequently administered in aerosol formulations through the mouth or nose. One widely used method for dispensing such aerosol drug formulations involves formulating the drug as a suspension or a solution in a liquefied gas propellant. The suspension/solution is stored in a sealed canister capable of withstanding the pressure required to maintain the propellant as a liquid. The suspension/solution is dispersed by activation of a dose-metering valve affixed to the canister.

A metering valve generally comprises a metering chamber, which is of a set volume and is designed to administer per actuation an accurate predetermined dose of medicament. The suspension/solution is forced from the dose-metering valve on actuation by the high vapour pressure of the propellant. The propellant rapidly vaporises leaving a cloud of very fine particles of the drug formulation. This cloud of particles is directed into the nose or mouth of the patient by a channelling device such as a cylinder or open-ended cone. Concurrently with the actuation of the aerosol dose-metering valve, the patient inhales the drug particles into the lungs or nasal cavity. Systems for dispensing drugs in this way are known as “metered dose inhalers” (MDIs). See Peter Byron, Respiratory Drug Delivery, CRC Press, Boca Raton, Fla. (1990) for a general background on this form of therapy.

Patients often rely on medication delivered by MDIs for rapid treatment of respiratory disorders, which are debilitating and in some cases even life threatening. Therefore, it is essential that the prescribed dose of aerosol medication delivered to the patient consistently meets the specifications claimed by the manufacturer and, complies with the requirements of the FDA and other regulatory authorities. That is, every dose delivered from an MDI must lie within a very tightly controlled specification range.

A problem which can exist with drug delivery devices such as MDIs is the is deposition of the medicament, or the solid component from a suspension of a particulate product in a liquid propellant, onto the internal surfaces of the device which can occur either immediately after manufacture or after a number of operation cycles and/or storage. This can lead to a reduction in the efficacy of the device and of the resulting treatment as the deposition of the product reduces the amount of active drug available to be dispensed to the patient and can also reduce the uniformity of the dose dispensed during the lifetime of the device.

The problem of drug adherence and dose uniformity can be greater with suspension formulations comprising hydrofluoroalkane propellants, for example, 1,1,1,2-tetrafluoroethane (HFA134a) and 1,1,1,2,3,3,3-n-heptafluoropropane (HFA227) which have been developed as ozone friendly replacements of chlorofluorocarbon propellants such as P11, P114 and P12.

Some conventional devices rely on shaking the dispenser to agitate the liquid propellant and product mixture therein, in an attempt to dislodge the deposited particles. However, while in some cases this remedy can be effective within the body of the drug canister itself, it may not be effective for particles deposited on the inner surfaces of the other MDI components, such as the metering valve.

One solution to this problem is to provide a coating on the internal surfaces of the valve or canister which contact the medicament and which inhibits drug deposition, wherein the coating is of a fluorinated polymer, for example PTFE. The process used to date to provide such a coating is a continuous wave radio frequency plasma process, such as a cold plasma radio frequency process, which in theory operates in an energy range of 2 MHz to 200 MHz, but practically is only able to operate at an energy of 13.56 MHz, 27.12 MHz and 40.68 MHz, since these are the only frequencies designated to industrial use in unrestricted power. There are, however, problems with using such a continuous wave radio frequency plasma process: the substrate and/or monomer may be damaged by the use of continuous waves of this specific energy and frequency; the inside of intricate shapes will not be coated or be insufficiently coated; some of the coating may break free and end up in the canister along with the drug formulation; and the coating may be difficult to characterise.

### SUMMARY OF THE INVENTION

It is therefore an aim of the present invention to provide a dispenser for a medicament in which one or more of the surfaces thereof have a fluorinated coating provided by a process which does not result in the problems mentioned above.

A further aim of the invention is to provide a coating which reduces moisture ingress into a pharmaceutical aerosol formulation, reduces the absorption of the drug into the substrate, e.g. of rubber, and reduces friction between movable parts in inhalation devices.

### DETAILED DESCRIPTION OF THE INVENTION

In a first aspect of the invention there is provided a medicament dispenser comprising a canister and a drug dispensing valve, wherein one or more surfaces of said canister and/or valve has a fluorinated coating provided by a process comprising generating one or more fluorine-containing radical species and polymerising said radicals on said one or more surfaces, provided that the radicals are not generated by a continuous wave radio frequency plasma process.

The invention also provides a process for the preparation of a medicament dispenser as hereinbefore defined, said process comprising generating one or more fluorine-containing radical species, and polymerising said radicals on said one or more surfaces, provided that the radical species are not generated by a continuous wave radio frequency plasma process. Polymerisation of the radicals may occur subsequent to generation of the radicals, or polymerisation may begin while additional radicals are being generated.

The radicals for use in such a process may be generated by a number of processes including, but not limited to, a plasma process which is not a continuous wave radio frequency (RF) plasma process, such as a microwave plasma process or a pulsed wave plasma process.

Microwave plasma process may be carried out in a continuous or a pulsed manner at an energy range of 300 MHz to 300 GHz, for example at energy of 2.45 GHz. A microwave process is a far more efficient process than the conventionally used continuous RF process and consequently the quantity of monomer gas required to provide equivalent coating is less. Furthermore, the coating times required are of the order of seconds as opposed to minutes or even hours for continuous RF triggered plasma process. Accordingly, any potential damage to the substrate is remarkably reduced. Non-fluorinated monomers can be used in addition to fluorinated monomers, the ratio of non-fluorinated to fluorinated either being constant throughout the process or varying. Suitably, the ratio is high at the beginning of the process and low at the end. Preferably, pure non-fluorinated monomer is used at the beginning of the process and pure fluorinated monomer used at the end of the process. In other words, a fluorinated monomer gas (e.g. fluorine gas) may be used as the medium from which the fluorine-containing radical species are generated either in isolation or mixed with a non-fluorinated monomer gas (mixed monomer gas). With a mixed monomer gas, the relative amounts of the monomers can be fixed or varied in the process, as outlined already above.

A pulsed wave plasma process provides advantages over the traditionally used continuous RF plasma processes. The pulsed process is more controllable since the average energy is lower. This results in less damage to the substrate and more control over the radical polymerisation. Pulses are applied on and off at intermittent intervals, suitably the ‘off’ time being longer than the ‘on’ time. Suitably, the ratio of ‘off’ time to ‘on’ time is such that the average power applied is less than 10 Watts, preferably less than 1 W. For example, the power may be applied for 20 μS, followed by no power for 10000 μS to 20000 μS. Furthermore, the ‘off’ time allows the radicals to penetrate into intricate substrates providing an improved coating for more intricate substrates. The energy applied may be in the microwave or RF wave range.

Ordinarily, the surface(s) to be coated is a surface which, in use of the dispenser, contacts the medicament, e.g. when the dispenser is filled with a medicament in a fluid propellant.

One advantage of using a coating process hereinbefore described is that intricate shapes may be coated and the coating is not restricted to the “line of sight”. That is, the inside of components not readily visible may be coated.

Suitably, the surface energy of the resulting coating of the invention, gives a contact angle of greater than 70 degrees, preferably greater than 90 degrees, more preferably greater than 110 degrees. The term “contact angle” is the angle between a liquid water droplet and the coated surface of the canister/valve at the liquid/solid interface as measured in ambient conditions, i.e. at a temperature of 20° C. (±5° C.) and a relative humidity of 50% (±20%). The contact angle may be measured on a coating deposited on a flat polybutylene terephthalate (PBT) substrate surface in accordance with the invention.

In a further embodiment, the dispenser of the invention further comprises moisture-absorbing means. The moisture absorbing means will generally comprise a desiccant material.

In one embodiment, the moisture absorbing means is contained within the canister. Preferably, the moisture absorbing means will be particulate, the particles being of a size which are not inhaled into the lung, for example they have a mean size (e.g. mass median diameter MMD) of greater than 100 μm. According to another aspect of this embodiment, preferably the moisture absorbing means is not able to pass through the valve (e.g. not able to enter the metering chamber of the valve), for example by virtue of its size. In one example of this arrangement, the moisture absorbing means is present in the canister as a tablet or bead. In an alternative aspect the moisture absorbing means is not able to pass through the valve because it is attached to the canister.

Examples of moisture absorbing means suitable for use include nylon. Another example is silica gel. Other exemplary moisture absorbing means include inorganic materials such as zeolites, alumina, bauxite, anhydrous calcium sulphate, water-absorbing clay, activated bentonite clay, a molecular sieve, or other like materials. When nylon is used it is preferably supplemented with use of another desiccant material having a higher water capacity (such as one of the inorganic materials just mentioned).

The moisture absorbing means should be present in sufficient quantity to absorb undesired moisture and will typically have a water absorption capacity of 20-50 weight percent. Typically 100 μg to 5 g, for example 1 mg to 5 g, e.g. 100 mg to 500 mg such as about 100 mg to 250 mg of moisture absorbing material should be adequate for a typical metered dose inhaler.

In a further embodiment of the invention, the canister and/or valve is partially or wholly manufactured of or incorporates a moisture absorbing means, suitably a desiccant material. Preferably, the material from which the canister and/or valve is manufactured will be loaded with at least 5% by weight of the moisture absorbing means, more preferably 10 to 80% by weight, especially 20 to 60% by weight. One embodiment is an acetal valve loaded with a desiccant material which is a molecular sieve.

Loading when used in this specification will be understood to include coating and/or lining. However, desiccant which is loaded may be adsorbed at least in part into the material from which the component is manufactured.

Preferably, the moisture absorbing means is incorporated within the valve rather than within the canister.

When the valve is a metering valve comprising a valve body which defines a metering chamber, the moisture absorbing means may, for example, be incorporated within the metering chamber of the valve. For example, the metering chamber may be partially, or preferably, wholly manufactured of nylon which is a natural desiccant material. Alternatively, the metering chamber may be coated with a moisture absorbing means.

The moisture absorbing means may instead (or in addition) be incorporated within one or more valve seals. As used herein, “valve seal” includes one or more of the following lower stem seal and/or upper stem seal and gasket seal employed in the valve for sealing purposes, generally composed of elastomeric materials.

In conjunction with the moisture absorbing means, e.g. desiccant, an additional compound may be added to act as a conduit/channelling agent to increase/optimise the efficiency of the moisture absorption properties. Such materials may include compounds such as polyethylene glycols.

Any parts of the canister and/or valve, which contact the pharmaceutical aerosol suspension, may be coated with the fluorinated coating of the invention. Such a coating reduces or eliminates the tendency of medicament particles to deposit or precipitate out thereon. Where the valve part is a movable part (e.g. the valve stem) the coating also reduces the friction between that part and another part of the valve (e.g. the stem seal). Accordingly, a further aspect of the invention provides a method of preventing drug deposition in a dispenser for dispensing a medicament in a fluid propellant having a canister for housing the medicament and a drug-dispensing valve, the method comprising the use of a dispenser as defined above.

In a further aspect, the invention provides a canister for housing the medicament, wherein one or more of the surfaces of said canister comprise a fluorinated coating, wherein said coating is prepared by a process as hereinbefore described.

Typically, the canister contains a pharmaceutical aerosol formulation comprising a medicament and a fluorocarbon propellant.

In another aspect, the invention provides a drug-dispensing valve for use in a dispenser for dispensing a medicament in a fluid propellant, wherein one or more of the surfaces of said valve comprise a fluorinated coating, prepared by a process as hereinbefore described.

Typically, the drug-dispensing valve is a drug metering valve.

The valve suitably comprises a number of components or parts, as known in the art. All may independently of the other components be coated with a fluorinated coating as hereinbefore defined. Component parts of the valve which may be coated include, but are not limited to, the metering chamber, valve stem, the upper and lower stem seals, neck gasket, spring and body. Suitably, the valve is a metering valve.

Thus another aspect of the invention provides a metering chamber, wherein one or more surfaces thereof comprise a fluorinated coating according to the present invention.

A further aspect of the invention provides a valve stem coated with a fluorinated coating according to the present invention. Such a coating on the valve stem may reduce its frictional contact properties, and the need for any further stem lubricant such as silicone oil is reduced or eliminated. Reducing frictional contact can be particularly advantageous where the valve is employed in a dispenser for both suspension and solution medicament formulations.

In order to improve adhesion of the fluorinated coating to the one or more surfaces, the surfaces to be coated may be subjected to a pre-treatment step to remove any surface contamination and/or to activate the surface. Accordingly, a further aspect of the invention provides a dispenser comprising a canister for housing the medicament and a drug dispensing valve, wherein one or more of the internal surfaces of the canister and/or valve comprises a fluorinated coating prepared by the process as hereinbefore defined, characterised in that the one or more of the internal surfaces of the canister and/or valve are provided with a pre-treatment step to remove surface contamination and/or to activate the surface. Additionally, there is provided a process for the preparation of a medicament dispenser as hereinbefore defined, said process comprising providing the one or more of the internal surfaces of the canister and/or valve with a pre-treatment step to remove surface contamination and/or to activate the surface, followed by providing a fluorinated coating on the one or more internal surfaces of the canister and/or valve, wherein the coating is prepared by a process hereinbefore defined.

Where the fluorinated coating is prepared by the formation of fluorine-containing radicals and the polymerisation thereof, the pre-treatment may be achieved by for example treatment of the components with an etching gas such as oxygen or argon. Preferably, the etching gas is oxygen. In the process, radicals react with the plastic or metal substrate; for example the component is exposed to a low pressure oxygen plasma environment which creates polar groups on the component's surface which are more conducive to bonding with the plasma coating to be applied.

The pre-treatment step, for example with oxygen, could be carried out under a range of conditions and duration. However, the Applicant has found that the following conditions provide a satisfactory pre-treatment: run time 120 seconds; power 400 W; gas pressure 300 mTorr; gas flow 80 cc/min; tumbler speed 35 rpm. It should be noted, however, that the invention is not limited to these conditions and that any set of conditions used for a pre-treatment step is within the scope of the invention.

The dispenser as hereinbefore defined is suitably incorporated as part of a “metered dose inhaler” or “MDI”. The term “metered dose inhaler” or “MDI” means a unit comprising a canister, a cap (ferrule) covering the mouth of the canister (typically crimped), a drug metering valve situated in the cap, a metering chamber and a suitable channelling device (actuator) into which the canister is fitted. The term “drug metering valve” or “MDI valve” refers to a valve and its associated mechanisms which deliver a predetermined amount of drug formulation from an MDI upon each activation. The channelling device may comprise, for example, an actuating device for the valve and a cylindrical or cone-like passage through which medicament may be delivered from the filled MDI can via the MDI valve to the nose or mouth of a patient, e.g. a mouthpiece actuator. The relation of the parts of a typical MDI is illustrated in U.S. Pat. No. 5,261,538, the content of which is hereby incorporated herein by reference. The metered dose inhalers may be prepared by methods of the art (e.g. see Byron above and U.S. Pat. No. 5,345,980, the contents of which are hereby incorporated herein by reference).

Thus, in another aspect, the invention provides a metered dose inhaler for dispensing a medicament in a fluid propellant comprising a dispenser as defined above and a medicament channelling device, such as an actuator.

In addition to the canister and the valve being provided with a fluorinated coating of the invention, other component parts of the MDI may also be provided with such a coating. Accordingly, a further aspect of the invention provides a ferrule having one or more of its surfaces provided with a fluorinated coating of the invention. A yet further aspect provides an actuator having one or more of its surfaces provided with a fluorinated coating of the invention.

Suitably, the entire valve or one or more of the valve components are made of a non-metal material. Suitable non-metals for use in the valve include pharmacologically resilient polymers such as acetal, polyamide (e.g. Nylon®), polycarbonate, polyester (e.g. polybutylterephthalate (PBT)), fluorocarbon polymer (e.g. Teflon®) or a combination of these materials. Additionally, seals and “O” rings of various materials (e.g., nitrile rubbers, polyurethane, acetyl resin, fluorocarbon polymers), or other elastomeric materials are employed in and around the valve. Alternatively, the valve is made of metal, for example stainless steel, aluminium, copper, tin plate and any alloys thereof.

The valve can have any suitable configuration. Metal and non-metal parts can be combined to optimise the performance of the valve.

Conventionally, the canisters and caps for use in MDIs are made of aluminium or an alloy of aluminium although other metals not affected by the drug formulation, such as stainless steel, an alloy of copper, or tin plate, may be used. An MDI canister may also be fabricated from glass or plastics. Preferably, however, the MDI canisters and caps employed in the present invention are made of aluminium or an alloy thereof.

The canister is preferably a pressurised container comprising an aluminium metal vial having a metering valve disposed therein. While the pressurised container preferably includes a metering valve, other valve systems are not beyond the scope of the present invention. Other valve systems include, but are not limited to, wedge gate valve systems, double-disc gate valve systems, globe and angle valve systems, swing check valve systems, end cock valve systems, and other like valve systems, as known in the art. Since the pressurised container is preferably part of an MDI, the valve design is typically a function of providing a predetermined dosage or amount of the drug contained within the pressurised container to a user.

The valve typically comprises a valve body having an inlet port through which the pharmaceutical aerosol formulation may enter said valve body, an outlet port through which the pharmaceutical aerosol may exit the valve body and an open/close mechanism by means of which flow through said outlet port is controllable.

The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

The valve may be a metering valve in which the valve body has a metering chamber, a sampling chamber and therebetween a second sealing ring within which the stem is slidably movable, the valve stem having a transfer passage such that in the valve-closed position the dispensing passage is isolated from the metering chamber and the metering chamber is in communication with the sampling chamber via the transfer passage, and in the valve-open position the dispensing passage is in communication with the metering chamber and the transfer passage is isolated from the metering chamber. The metering volumes are typically from 50 to 100 μl, such as 50 μl or 63 μl.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined therebetween and such that during movement between its non-dispensing and dispensing positions the valve stem sequentially: (i) allows free flow of aerosol formulation into the chamber, (ii) defines a closed metered volume for pressurised aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurised aerosol formulation. A valve of this type is described in U.S. Pat. No. 5,772,085 the content of which is hereby incorporated herein by reference.

The valve may also have a structure and action similar to those aerosol valves described in European Patent Application No. EP-A-870,699 and PCT Patent Application No. WO99/36334, the contents of which are hereby incorporated herein by reference.

The neck gasket (sealing ring) may be formed by cutting a ring from a sheet of suitable material. Alternatively, the neck gasket may be formed by a moulding process such as an injection moulding, a compression moulding or a transfer moulding process.

Typically, the neck gasket(s) comprises an elastomeric material. The ring is typically resiliently deformable.

The elastomeric material may either comprise a thermoplastic elastomer (TPE) or a thermoset elastomer, which may optionally be cross-linked. The sealing ring may also comprise a thermoplastic elastomer blend or alloy in which an elastomeric material is dispersed in a thermoplastic matrix. The elastomers may optionally additionally contain conventional polymer additives such as processing aids, colorants, tackifiers, lubricants, silica, talc, or processing oils such as mineral oil in suitable amounts.

Suitable thermoset rubbers include butyl rubbers, chloro-butyl rubbers, bromo-butyl rubbers, nitrile rubbers, silicone rubbers, fluorosilicone rubbers, fluorocarbon rubbers, polysulphide rubbers, polypropylene oxide rubbers, isoprene rubbers, isoprene-isobutene rubbers, isobutylene rubbers or neoprene (polychloroprene) rubbers.

Suitable thermoplastic elastomers comprise a copolymer of about 80 to about 95 mole percent ethylene and a total of about 5 to about 20 mole percent of one or more comonomers selected from the group consisting of 1-butene, 1-hexene, and 1-octene as known in the art. Two or more such copolymers may be blended together to form a thermoplastic polymer blend.

Another suitable class of thermoplastic elastomers are the styrene-ethylene/butylene-styrene block copolymers. These copolymers may additionally comprise a polyolefin (e.g. polypropylene) and a siloxane.

Thermoplastic elastomeric material may also be selected from one or more of the following: polyester rubbers, polyurethane rubbers, ethylene vinyl acetate rubber, styrene butadiene rubber, copolyether ester TPE, olefinic TPE, polyester amide TPE and polyether amide TPE.

Other suitable elastomers include ethylene propylene diene rubber (EPDM). The EPDM may be present on its own or present as part of a thermoplastic elastomer blend or alloy, e.g. in the form of particles substantially uniformly dispersed in a continuous thermoplastic matrix (e.g. polypropylene or polyethylene). Commercially available thermoplastic elastomer blend and alloys include the SANTOPRENE™ elastomers. Other suitable thermoplastic elastomer blends include butyl-polyethylene (e.g. in a ratio ranging between about 2:3 and about 3:2) and butyl-polypropylene.

Typically, the stem gasket(s) additionally comprises and/or are coated with lubricant material.

In addition, the stem may also comprise lubricant material. Suitably, the valve stem comprises up to 30% by weight, preferably from 5 to 20% by weight of lubricant material.

The term ‘lubricant’ herein means any material that reduces friction between the valve stem and seal. Suitable lubricants include silicone oil or a fluorocarbon polymer such as polytetrafluoroethane (PTFE) or fluoroethylene propylene (FEP).

Lubricant can be applied to the stem, or stem gasket(s) by any suitable process including coating and impregnation, such as by injection or a tamponage process.

In medical use the canisters in accordance with the invention may contain a pharmaceutical aerosol formulation comprising a medicament and a fluorocarbon or hydrogen-containing chlorofluorocarbon propellant.

Suitable propellants include, for example, C₁₋₄hydrogen-containing chlorofluorocarbons such as CH₂ClF, CClF₂CHCIF, CF₃CHClF, CHF₂CClF₂, CHClFCHF₂, CF₃CH₂Cl and CClF₂CH₃; C₁₋₄hydrogen-containing fluorocarbons such as CHF₂CHF₂, CF₃CH₂F, CHF₂CH₃ and CF₃CHFCF₃; and perfluorocarbons such as CF₃CF₃ and CF₃CF₂CF₃.

Where mixtures of the fluorocarbons or hydrogen-containing chlorofluorocarbons are employed they may be mixtures of the above identified compounds or mixtures, preferably binary mixtures, with other fluorocarbons or hydrogen-containing chlorofluorocarbons for example CHClF₂, CH₂F₂ and CF₃CH₃. Preferably a single fluorocarbon or hydrogen-containing chlorofluorocarbon is employed as the propellant. Particularly preferred as propellants are C₁₋₄hydrogen-containing fluorocarbons such as 1,1,1,2-tetrafluoroethane (CF₃CH₂F) and 1,1,1,2,3,3,3-heptafluoro-n-propane (CF₃CHFCF₃) or mixtures thereof.

The pharmaceutical formulations for use in the canisters of the invention contain no components that provoke the degradation of stratospheric ozone. In particular the formulations are substantially free of chlorofluorocarbons such as CCl₃F, CCl₂F₂ and CF₃CCl₃.

The propellant may additionally contain a volatile adjuvant such as a saturated hydrocarbon for example propane, n-butane, isobutane, pentane and isopentane or a dialkyl ether for example dimethyl ether. In general, up to 50% w/w of the propellant may comprise a volatile hydrocarbon, for example 1 to 30% w/w. However, formulations, which are free or substantially free of volatile adjuvants are preferred. In certain cases, it may be desirable to include appropriate amounts of water, which can be advantageous in modifying the dielectric properties of the propellant.

The invention is particularly useful with propellants (including propellant mixtures) which are more hygroscopic than P11, P114 and/or P12 such as HFA-134a and HFA-227.

A polar co-solvent such as C₂₋₆ aliphatic alcohols and polyols e.g. ethanol, isopropanol and propylene glycol, preferably ethanol, may be included in the drug formulation in the desired amount to improve the dispersion of the formulation, either as the only excipient or in addition to other excipients such as surfactants. Suitably, the drug formulation may contain 0.01 to 30% w/w based on the propellant of a polar co-solvent e.g. ethanol, preferably 0.1 to 20% w/w e.g. about 0.1 to 15% w/w. In aspects herein, the solvent is added in sufficient quantities to solubilise a part or all of the medicament component, such formulations being commonly referred to as solution formulations.

A surfactant may also be employed in the aerosol formulation. Examples of conventional surfactants are disclosed in EP-A-372,777; the content of which is hereby incorporated herein by reference. The amount of surfactant employed is desirable in the range 0.0001% to 50% weight to weight ratio relative to the medicament, in particular, 0.05 to 5% weight to weight ratio.

The final aerosol formulation desirably contains 0.005-10% w/w, preferably 0.005 to 5% w/w, especially 0.01 to 1.0% w/w, of medicament relative to the total weight of the formulation.

Medicaments which may be administered in the aerosol formulations include any drug useful in inhalation therapy. The dispenser of the invention is in one aspect suitable for dispensing medicament for the treatment of respiratory disorders such as disorders of the lungs and bronchial tracts including asthma and chronic obstructive pulmonary disorder (COPD). In another aspect, the invention is suitable for dispensing medicament for the treatment of a condition requiring treatment by the systemic circulation of medicament, for example migraine, diabetes, pain relief, e.g. with inhaled morphine.

Accordingly, in one aspect of the invention, there is provided the use of a dispenser or MDI according to the invention for the treatment of a respiratory disorder, such as asthma and COPD. Additionally, the present invention provides a method of treating a respiratory disorder such as, for example, asthma and COPD, which comprises administration by inhalation of an effective amount of an aerosol formulation as herein described from a dispenser or MDI of the present invention.

A further aspect of the invention provides the use of a dispenser or MDI according to the invention for the treatment of a condition requiring the systemic circulation of a medicament, such as, for example, migraine, diabetes, chronic pain.

The present invention also provides a method of treating a condition requiring the systemic circulation of medicament, such as, for example migraine, diabetes and chronic pain, which comprises administration by inhalation of an effective amount of an aerosol formulation as herein described from a dispenser or MDI or the present invention.

Appropriate medicaments may thus be selected from, for example, analgesics, e.g., codeine, dihydromorphine, ergotamine, fentanyl or morphine; anginal preparations, e.g., diltiazem; antiallergics, e.g., cromoglycate (e.g. s the sodium salt), ketotifen or nedocromil (e.g. as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (e.g. as the dipropionate ester), fluticasone (e.g. as the propionate or furoate ester), flunisolide, budesonide, rofleponide, mometasone e.g. as the furoate ester), ciclesonide, triamcinolone (e.g. as the acetonide) or 6α,9α-difluoro-11β-hydroxy-16α-methyl-3-oxo-17α-propionyloxy-androsta-1,4-diene-17β-carbothioic acid S-(2-oxo-tetrahydro-furan-3-yl)ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]ethyl-2(3H)-benzothiazolone; adenosine 2a agonists, e.g. 2R,3R,4S,5R)2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate); α₄ integrin inhibitors e.g. (2S)-3-[4-({[4-(aminocarbonyl)-1-piperidinyl]carbonyl}oxy)phenyl]-2-[((2S)-4-methyl-2-{[2-(2-methylphenoxy)acetyl]amino}pentanoyl)amino]propanoic acid (e.g. as free acid or potassium salt), diuretics, e.g., amiloride; anticholinergics, e.g., ipratropium (e.g. as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; vaccines, diagnostics, and gene therapies. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament.

Preferred medicaments are selected from albuterol, salmeterol, fluticasone propionate and beclomethasone dipropionate and salts or solvates thereof, e.g., the sulphate of albuterol and the xinafoate of salmeterol.

Medicaments can also be delivered in combinations. Preferred formulations containing combinations of active ingredients contain salbutamol (e.g., as the free base or the sulphate salt) or salmeterol (e.g., as the xinafoate salt) or formoterol (e.g. as the fumarate salt) in combination with an anti-inflammatory steroid such as a beclomethasone ester (e.g., the dipropionate) or a fluticasone ester (e.g., the propionate) or budesonide. A particularly preferred combination is a combination of fluticasone propionate and salmeterol, or a salt thereof (particularly the xinafoate salt). A further combination of particular interest is budesonide and formoterol (e.g. as the fumarate salt).

Particularly preferred formulations for use in the canisters of the present invention comprise a medicament and a C₁₋₄ hydrofluoroalkane particularly 1,1,1,2-tetrafluoroethane and 1,1,1,2,3,3,3-n-heptafluoropropane or a mixture thereof as propellant.

Conventional bulk manufacturing methods and machinery well known to those skilled in the art of pharmaceutical aerosol manufacture may be employed for the preparation of large scale batches for the commercial production of filled canisters. Thus, for example, in one bulk manufacturing method a metering valve is crimped onto a can to form an empty canister. The particulate medicament is added to a charge vessel and liquefied propellant is pressure filled through the charge vessel into a manufacturing vessel. The drug suspension is mixed before re-circulation to a filling machine and an aliquot of the drug suspension is then filled through the metering valve into the canister. Typically, in batches prepared for pharmaceutical use, each filled canister is check-weighed, coded with a batch number and packed into a tray for storage before release testing.

Each filled canister is conveniently fitted into a suitable channelling device prior to use to form a metered dose inhaler for administration of the medicament into the lungs or nasal cavity of a patient. Suitable channelling devices comprise for example a valve actuator and a cylindrical or cone-like passage through which medicament may be delivered from the filled canister via the metering valve to the nose or mouth of a patient e.g., a mouthpiece actuator. Metered dose inhalers are designed to deliver a fixed unit dosage of medicament per actuation or “puff”, for example in the range of 2 to 5000 microgram medicament per puff.

Administration of medicament may be indicated for the treatment of mild, moderate or severe acute or chronic symptoms or for prophylactic treatment. It will be appreciated that the precise dose administered will depend on the age and condition of the patient, the particular particulate medicament used and the frequency of administration and will ultimately be at the discretion of the attendant physician. When combinations of medicaments are employed the dose of each component of the combination will in general be that employed for each component when used alone. Typically, administration may be one or more times, for example from 1 to 8 times per day, giving for example 1, 2, 3 or 4 puffs each time. Each valve actuation, for example, may deliver 5 μg, 50 μg, 100 μg, 200 μg or 250 μg of a medicament. Typically, each filled canister for use in a metered dose inhaler contains 60, 100, 120 or 200 metered doses or puffs of medicament; the dosage of each medicament is either known or readily ascertainable by those skilled in the art.

It will be understood that the present disclosure is for the purpose of illustration only and the invention extends to modifications, variations and improvements thereto which will be within the ordinary skill of the person skilled in the art.

1. A medicament dispenser comprising a canister and a drug dispensing valve, wherein one or more surfaces of said canister and/or valve has a fluorinated coating provided by a process comprising generating one or more fluorine-containing radical species and polymerising said radicals on said one or more surfaces, provided that the radicals are not generated by a process involving continuous wave radio frequency plasma polymerisation.

2. A medicament dispenser according to claim 1 in which the process is a plasma process.

3. A medicament dispenser according to claim 1, wherein the fluorine-containing radical species are generated by a microwave plasma process.

4. A medicament dispenser according to claim 1, wherein the fluorine-containing radical species are generated by a pulsed wave plasma process.

5. A medicament dispenser according to claim 1 wherein the fluorine-containing radical species are produced from a fluorinated gas.

6. A medicament dispenser according to claim 5 wherein the gas comprises an inert gas.

7. A medicament dispenser according to claim 5, wherein the gas comprises fluorine gas.

8. A medicament dispenser according to claim 1, wherein the fluorine-containing radial species are produced from a gas mixture containing fluorine and non-fluorine components.

9. A medicament dispenser according to claim 8 in which the ratio of fluorine to non-fluorine components in the gas is varied during the process.

10. A medicament dispenser according to claim 8 in which the ratio of fluorine to non-fluorine components in the gas is maintained constant, or maintained substantially constant, during the process.

11. A medicament dispenser according to any preceding claim, wherein the fluorinated coating gives a contact angle of greater than 70 degrees.

12. A medicament dispenser according to claim 1, wherein the dispenser further comprises moisture absorbing means.

13. A medicament dispenser according to claim 12, wherein the canister and/or valve is partially or wholly manufactured of or incorporates the moisture absorbing means.

14. A canister for housing a medicament, wherein one or more surfaces of said canister comprises a fluorinated coating provided by a process comprising generating one or more fluorine-containing radical species and polymerising said radicals on said one or more surfaces, provided that the radicals are not generated by a process involving continuous wave radio frequency plasma polymerisation.

15. A drug-dispensing valve for use in a dispenser for dispensing a medicament in a fluid propellant, wherein one or more surfaces of said valve comprise a fluorinated coating provided by a process comprising generating one or more fluorine-containing radical species, polymerising said radicals on said one or more surfaces, provided that the radicals are not generated by a process involving continuous wave radio frequency plasma polymerisation.

16. A drug-dispensing valve according to claim 15, wherein the drug-dispensing valve is a drug-metering valve.

17. A drug-dispensing valve according to claim 15, wherein the fluorinated coating is provided on one or more valve components selected from the group consisting of a metering chamber, valve stem, upper stem seal, lower stem seal, neck gasket, spring and body.

18. A drug-dispensing valve according to claim 15, wherein the fluorinated coating is provided on one or more surfaces of a metering chamber.

19. A metering chamber, wherein one or more surfaces thereof are provided with a fluorinated coating provided by a process comprising generating one or more fluorine-containing radical species and polymerising said radicals on said one or more surfaces, provided that the radicals are not generated by a process involving continuous wave radio frequency plasma polymerisation.

20. A valve stem, wherein one or more surfaces thereof are provided with a fluorinated coating provided by a process comprising generating one or more fluorine-containing radical species and polymerising said radicals on said one or more surfaces, provided that the radicals are not generated by a process involving continuous wave radio frequency plasma polymerisation.

21. A medicament dispenser according to claim 1, wherein the one or more surfaces are provided with a pre-treatment step prior to being provided with the fluorinated coating to remove surface contamination and/or to activate the surface.

22. A metered dose inhaler for dispensing a medicament in a fluid propellant, comprising a dispenser according to claim 1, and a medicament channelling device.

23. A process for producing a medicament dispenser according to claim 1 comprising the steps of:
(a) generating one or more fluorine-containing radical species, and
(b) polymerising said radicals on said one or more surfaces, provided that the radical species are not generated by a continuous wave radio frequency plasma process.

24. A method of treating a respiratory disorder comprising:
(a) providing a patient having a respiratory disorder a medicament dispenser according to claim 1 and
(b) delivering a medicament from said medicament dispenser to the patient.

25. A method of treating a respiratory disorder, comprising administering by inhalation an effective amount of medicament in a fluid propellant from a dispenser according to claim 1.

26. A method of treating a condition in a patient requiring systemic circulation of a medicament comprising the steps of:
(a) providing to said patient a medicament dispenser according to claim 1 having a medicament contained therein, and
(b) administering said medicament to said patient.

27. A method of treating a condition requiring systemic circulation of a medicament, comprising administering by inhalation an effective amount of medicament in a fluid propellant from a dispenser according to claim 1.

28. A canister according to claim 14 wherein the one or more surfaces are provided with a pre-treatment step prior to being provided with the fluorinated coating to remove surface contamination and/or to activate the surface.

29. A valve according to claim 15, wherein the one or more surfaces are provided with a pre-treatment step prior to being provided with the fluorinated coating to remove surface contamination and/or to activate the surface.

30. A metering chamber according to claim 19, wherein the one or more surfaces are provided with a pre-treatment step prior to being provided with the fluorinated coating to remove surface contamination and/or to activate the surface.

31. A valve stem according to claim 20, wherein the one or more surfaces are provided with a pre-treatment step prior to being provided with the fluorinated coating to remove surface contamination and/or to activate the surface.

32. A process for producing a canister according to claim 14, comprising the steps of:
(a) generating one or more fluorine-containing radical species, and
(b) polymerising said radicals on said one or more surfaces, provided that the radical species are not generated by a continuous wave radio frequency plasma process.

33. A process for producing a valve according to claim 15, comprising the steps of:
(a) generating one or more fluorine-containing radical species, and
(b) polymerising said radicals on said one or more surfaces, provided that the radical species are not generated by a continuous wave radio frequency plasma process.

34. A process for producing a metering chamber according to claim 19 comprising the steps of:
(a) generating one or more fluorine-containing radical species, and
(b) polymerising said radicals on said one or more surfaces, provided that the radical species are not generated by a continuous wave radio frequency plasma process.

35. A process for producing a valve stem according to claim 20, comprising the steps of:
(a) generating one or more fluorine-containing radical species, and
(b) polymerising said radicals on said one or more surfaces, provided that the radical species are not generated by a continuous wave radio frequency plasma process.

36. A method of treating a respiratory disorder comprising:
(a) providing to a patient having a respiratory disorder a metered dose inhaler according to claim 22 and
(b) delivering a medicament from said medicament dispenser to the patient.

37. A method of treating a respiratory disorder, comprising administering by inhalation an effective amount of medicament in a fluid propellant from a metered dose inhaler according to claim 22.

38. A method of treating a condition in a patient requiring systemic circulation of a medicament comprising the steps of:
(a) providing to said patient a metered dose inhaler according to claim 22 having a medicament contained therein, and
(b) administering said medicament to said patient.

39. A method of treating a condition requiring systemic circulation of a medicament, comprising administering by inhalation an effective amount of medicament in a fluid propellant from a metered dose inhaler according to claim 22.
